(19) United States
(12) Patent Application Publication
(10) Pub. No.: US 2003/0109029 A1
(43) Pub. Date: Jun. 12, 2003

(54) Process for the preparation of enantiomerically pure tertiary ss-hydroxycarboxylic acids or their esters

(76) Inventors: Alfred Popp, Unterhaching (DE); Hermann Petersen, Burghausen (DE); Juergen Stohrer, Pullach (DE); Jodoca Rockinger-Mechlem, Gilching (DE); Andrea Gilch, Mauern (DE)

Correspondence Address: BROOKS & KUSHMAN, 1000 TOWN CENTER 22ND FL, SOUTHFIELD, MI, 48075

(21) Appl. No.: 10/255,776

(22) Filed: Sep. 26, 2002

(30) Foreign Application Priority Data
Sep. 27, 2001 (DE) 101 47 653.1

(51) Int. Cl. C12P 41/00

(52) U.S. Cl. 435/280

(57) ABSTRACT

A process for the preparation of an enantiomerically pure tertiary β-hydroxycarboxylic acid or its ester, wherein an enantiomer mixture of compounds is brought into contact with an enzyme, which is capable of the hydrolytic cleavage of an ester bond, in an aqueous medium such that one enantiomer of the enantiomer mixture is hydrolyzed.

### CROSS-REFERENCE TO RELATED APPLICATION

This application claims the benefit of German application No. DE 101 47 653.1, filed Sep. 27, 2001, which is hereby incorporated by reference.

### BACKGROUND OF THE INVENTION 1. Field of the Invention

The invention relates to a process for the preparation of enantiomerically pure tertiary β-hydroxycarboxylic acids or esters.

2. Background Art

Enantiomerically pure derivatives are often used as starting materials or intermediates in the synthesis of agrochemicals and pharmaceuticals. Typically, these compounds are prepared and sold as racemic or diastereomeric mixtures. However, in many instances, a desired physiological effect is brought about by only one enantiomer or diastereomer. Although it is desirable that the other isomer be inactive, this is not always the case because the other isomer often counteracts the desired effect or is toxic. Accordingly, processes for the separation of racemates and diastereomers are important for the preparation of highly enantiomerically pure compounds.

It is known that the optical resolution of chiral compounds can be carried out with the aid of enzymes. Furthermore, kinetic resolution of esters with lipases and esterases are described in a number of publications. Generally, the resolution of secondary alcohols is accomplished by acylation of the hydroxyl group on the stereogenic center or by hydrolysis of the corresponding ester. If, a second functional group is present in such compounds, separation of the enantiomers can also be achieved by reaction with this second functional group. For example, the preparation of enantiomerically pure secondary α- or β-hydroxycarboxylic acids and enantiomerically pure tertiary α-hydroxycarboxylic acids has been described for when the second functional group is a carboxylic acid derivative.

EP 459455 (K. Miyazawa, K. et al.) describes a method for optically resolving secondary α-hydroxy esters by transesterification in the presence of a lipase prepared from Pseudomonas species under anhydrous conditions. EP 391345 (N. Murakami, et al.) describes a method for the optical separation of secondary β-hydroxy esters by stereoselective hydrolysis of the ester group in the presence of microorganisms. The processes of U.S. Pat. No. 5,643,793 (H. Hans), EP 736606 (A. Tixidre), and EP 494203 are all based on hydrolysis of either a desired or undesired steroisomer followed by separation and subsequent isolation of two optical antipodes. U.S. Pat. No. 5,643,793 discloses a preparation of enantiomerically pure 3-hydroxyhexanoic acid using porcine pancreas lipase (PPL). EP 736606 discloses a preparation of ethyl 4,4,4-trifluoro-3-(R)-hydroxybutanoate by hydrolysis of an ester group using an enzyme from Candida antarctica. Finally, EP494203 describes the separation of the optical isomers of secondary arylalkyl β-hydroxycarboxylic acid esters using Pseudomonas fluorescens lipase (PFL).

EP 512848 (Ch. Yee, et al.) and EP 786012 (F. Sariaslani, et al.) describe a method for the optical separation of tertiary β-hydroxycarboxylic acid esters. The method of Yee et al. is restricted to the use of an enzyme from Candida lipolytica, while Sariaslani et al. carry out the same reaction using a number of different hydrolytically active enzymes.

Although a number of methods exist for the preparation of enantiomerically pure tertiary α-hydroxy carboxylic acids and esters only a few studies describing the preparation of enantiomerically pure tertiary β-hydroxycarboxylic acids and esters are known. Specifically, the preparation of such compounds is restricted to two specific cases. The first method involves the desymmetrization of meso-diesters such as dimethyl β-hydroxy-β-methylglutarate (F. Huang et al. J. AM. CHEM. SOC. 1975, 97(14), pp. 4144-4145; E. Toone, et al. J. AM. CHEM. SOC. 1990, 112(12), pp. 4946-4952); and the second method involves enantioselective hydrolysis of 3-hydroxy-3-methylalkanoic acid esters using porcine liver esterase (PLE) (W. K. Wilson, et al. J. ORG. CHEM. 1983, 48 (22), pp. 3960-3966). The disadvantages of the latter method include extremely low selectivity (E=2.4-9), low enantiomeric purities of the products, and low chemical yields. For a definition of E see C. Chen et al. J. AM. CHEM. SOC. 1982, 104, pp. 7294-7299.

Accordingly, there exists a need for improved optical resolution processes. Such processes should be characterized by high enantiomeric purity of the optical antipodes; high chemical yield; high selectivity of the enzyme; good space-time yields; and inexpensive synthesis.

### SUMMARY OF THE INVENTION

The present invention overcomes the problems encountered in the prior art by providing an inexpensive process for the preparation of enantiomerically pure tertiary β-hydroxycarboxylic acids having formulae Ia and Ib or enantiomerically pure tertiary β-hydroxycarboxylic acid esters having formulae IIa and IIb. The process of the present invention is best understood by reference to Schemes I and II.

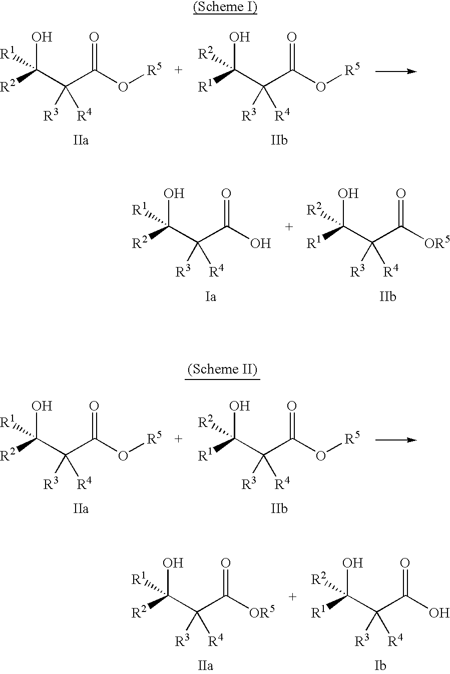

wherein R¹ and R² are each independently a substituted or unsubstituted (i.e., optionally substituted) C₆-C₁₈-aryl, C₃-C₁₈-heteroaryl, C₂-Cl₁₈-alkyl, C₂-C₁₈-alkenyl, C₂-C₁₈-alkynyl, C₆-C₁₈-aryl-C₁-C₁₈-alkyl, C₃-C₁₈-heteroaryl-C₁-C₁₈-alkyl, C₆-C₁₈-aryl-C₂-C₁₈-alkenyl, C₃-C₁₈-hetero-aryl-C₂-C₁₈-alkenyl, C₁-C₁₈-alkoxy-C₁-C₁₈-alkyl, C₁-C₁₈-alkoxy-C₂-C₁₈-alkenyl, C₆-C₁₈-aryloxy-C₁-C₁₈-alkyl, C₆-C₁₈-aryloxy-C₂-C₁₈-alkenyl, C₃-C₈-cycloalkyl, C₃-C₈-cyclo-alkyl-C₁-C₁₈-alkyl, C₃-C₈-cycloalkyl-C₂-C₁₈-alkenyl group with the proviso that R¹ and R² are not the same, or R¹ and R² together with the carbon to which they are bonded form a substituted, unsubstituted or heteroatom-containing cycloalkylidene, R³ and R⁴ are the same or different and each independently are substituted or unsubstituted C₆-C₁₈-aryl, C₃-C₁₈-heteroaryl, C₁-C₁₈-alkyl, C₂-C₁₈-alkenyl, C₂-C₁₈-alkynyl, C₆-C₁₈-aryl-C₁-C₁₈-alkyl, C₃-C₁₈-heteroaryl-C₁-C₁₈-alkyl, C₆-C₁₈-aryl-C₂-C₁₈-alkenyl, C₃-C₁₈-heteroaryl-C₂-C₁₈-alkenyl, C₁-C₁₈-alkoxy-C₁-C₁₈-alkyl, C₁-C₁₈-alkoxy-C₂-C₁₈-alkenyl, C₆-C₁₈-aryloxy-C₁-C₁₈-alkyl, C₆-C₁₈-aryloxy-C₂-C₁₈-alkenyl, C₃-C₈-cycloalkyl, C₃-C₈-cycloalkyl-C₁-C₁₈-alkyl, C₃-C₈-cycloalkyl-C₂-C₁₈-alkenyl group or R³ and R⁴, together with the carbon to which they are bonded form a substituted, unsubstituted or heteroatom-containing cycloalkylidene group, R⁵ is substituted or unsubstituted C₁-C₁₈-alkyl or C₂-C₁₈-alkenyl.

Preferred substituents for R¹, R², R³, R⁴, and any rings formed by joining R¹ and R² or R³ and R⁴ include alkyl, alkenyl, alkynyl, aryl, heteroaryl, hydroxyl, alkoxy, carboxylate, alkoxycarbonyl, amino, nitro or halogen. Furthermore, preferred heteratoms any of the optically substituent groups listed above include O, N or S.

### BRIEF DESCRIPTION OF THE DRAWINGS

FIG. 1 is a plot showing the progress of the hydrolysis of (rac)-methyl 3-hydroxy-5-phenyl-3-propyl-(E)-4-pentenoate (HSCMe) to (R)-(+)-methyl 3-hydroxy-5-phenyl-3-propyl-(E)-4-pentenoate (HSC).

FIG. 2 illustrates the synthetic scheme of the present invention for the preparation of tertiary β-hydroxy-carboxylic acids or esters having formula I and formula II.

### DETAILED DESCRIPTION OF THE PREFERRED EMBODIMENTS

The method of the present invention comprises bringing an enantiomeric mixture of compounds of the formula IIa and IIb into contact with an enzyme which is capable of the hydrolytic cleavage of an ester bond in an aqueous medium, such that one enantiomer of the enantiomer mixture is hydrolyzed.

The enantiomer mixture of compounds of the formula II is preferably an enantiomer mixture having formula III,

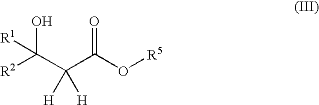

wherein R¹, R² and R⁵ are the same as set forth above. The enantiomeric mixture of formula II is most preferably an enantiomer mixture having formula IV:

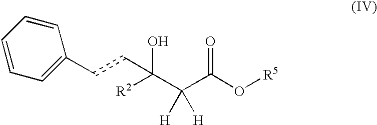

wherein R² and R⁵ have the meaning already mentioned and the dashed bond represents either a single or double bond.

Suitable enzymes for the process of the present invention include any enzyme capable of the cleavage an ester bond. Preferably, the enzyme is a lipase or esterase of class 3.1 according to International Enzyme Nomenclature, Committee of the International Union of Biochemistry and Molecular Biology. Lipases or esterases of microbial origin, porcine pancreas lipase or equine/porcine liver esterase are particularly preferred because each is readily accessible. Specifically, enzymes of microbial origin may be obtained from fungi, yeasts or bacteria such as, from Alcaligenes sp., Aspergillus niger, Aspergillus oryzae, Bacillus sp., Bacillus stearothermophilus, Bacillus thermoglucosidasius, Candida antarctica, Candida lipolytica, Candida rugosa, Chromobacterium viscosum, Geotrichum scandium, Mucor miehei, Penicillium camembertii, Penicillium roquefortii, Pseudomonas cepacia, Pseudomonas fluorescens, Pseudomonas sp., Rhizomucor javanicus, Rhizopus arrhizus, Rhizopus niveus, Saccharomyces cerevisiae, Thermoanaerobium brockii, and Thermomyces lanuginosa.

Lipases and esterases from Candida species such as Candida antarctica B and porcine liver esterase are particularly preferred. Most preferred are the enzymes Novozym® 435, 525 (Novo, Denmark) and Chirazyme® L2, E1, E2 (Böhringer Mannheim, Germany).

The enzymes are employed in the reaction directly or as immobilizates on all types of supports. The immobilizates can be prepared by dissolving the enzyme in a buffer at suitable pH and subsequent passive adsorption on a support such as diatomaceous earth (Celite®), activated carbon, alumina, silica gel, kieselguhr, monodisperse soluble organo-siloxane particles or resins (e.g. Amberlite®, Dowex®). Alternatively, the enzymes can also be covalently bonded to the support (e.g. polystyrene or epoxy resins such as Eupergit®). The supported enzymes can be dried by lyophilization.

The amount of enzyme to be added depends on the nature of the starting material, the product, and the activity of the enzyme preparation. The amount of enzyme optimal for the reaction can easily be determined by simple preliminary experiments. Depending on the enzyme, the enzyme-substrate ratio is calculated as the molar ratio between enzyme and substrate. This value is typically between 1:1,000 to 1:50,000,000, and preferably 1:10,000 to 1:5,000,000. The enantioselectivity E of the enzymes used in the present invention is typically between 5 and 100, or more. Preferably, the enantioselectivity is greater than 10.

The aqueous medium used for the hydrolysis reaction is preferably water. Preferably, the aqueous medium has a specified pH that is established by addition of a buffer. Most preferably, an Na₂HPO₄/NaH₂PO₄ buffer having a pH of 7.0 is used.

In order to keep the pH constant during the reaction, an aqueous alkali may also be added to the aqueous medium. An aqueous alkali is preferably the solution of an alkali metal hydroxide in water. The aqueous solution of NaOH or KOH is particularly preferred.

The enzyme reaction can be carried out without additional organic solvents or solvents, suspensions or emulsions of solvents in water, or buffers, as reaction mediums. Conventional emulsifiers may be used to improve the emulsion formation. However, additional solvents or solvent mixtures are preferably added to the reaction. Suitable solvents include aprotic or protic solvents. Such solvents should be inert with respect to the reaction of the present invention. Unsuitable solvents include for example, solvents that induce side reactions by acting as enzyme substrates (e.g. esters of primary and secondary alcohols). Suitable solvents include, but are not limited to, pure aliphatic or aromatic hydrocarbons such as hexane, cyclohexane, petroleum ether or toluene, halogenated hydrocarbons such as methylene chloride or chloroform; ethers such as methyl tert-butyl ether (MTBE), tetrahydrofuran, diethyl ether, diisopropyl ether or dioxane; tertiary alcohols such as tert-butanol, tert-pentyl alcohol; and esters of tertiary alcohols such as tert-butyl acetate or acetonitrile. Preferred solvents are methyl tert-butyl ether (MTBE) or diisopropyl ether.

The reaction of the present invention is preferably carried out at a temperature between 0° C. and 75° C. More preferably the reaction temperature is between 10° C. and 60° C., and most preferably between 20° C. and 50° C. Reaction times depend on the substrate, ester, and type and amount of enzyme. Typically, the reaction time is between 10 minutes and 7 days. Preferably, the reaction time is between 1 and 48 hours.

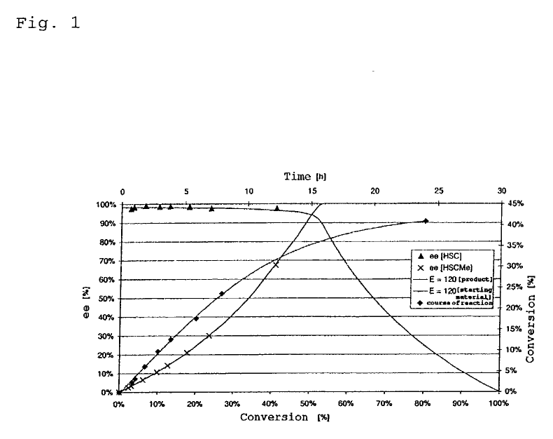

The course of the reaction can be monitored by methods known to those skilled in the art of optical resolution. Two such methods are the monitoring of alkali consumption during pH-stat titration or HPLC. The reaction is terminated depending on the desired result (i.e., high reaction, high enantiomer excess of the substrate or of the product, see FIG. 1). Ideally, the reaction is ended at a conversion of 50% with a high enantiomer purity both in the substrate and in the product (FIG. 1). The reaction is stopped by separating the unreacted enantiomer from the enantiomer mixture or by separating the product of the enzymatic reaction. Such a separation may be accomplished, for example, e.g. by extraction of the aqueous phase or distillation.

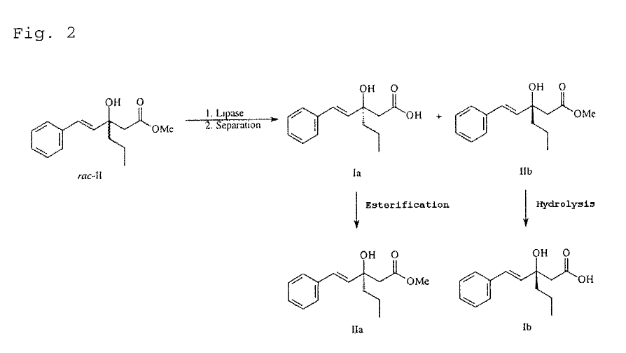

Depending on the enzyme, the (R)- or (S)-stereoisomer (see formula II: formula IIa or IIb) of the ester is hydrolyzed and the corresponding free acid (see formuala: formula Ia or Ib) is selectively formed. In each case the other enantiomer is unreacted and remains unchanged at the ester stage. For example, FIG. 2 shows, the synthesis for an enantiomer of the acid Ia and of the corresponding ester IIb having opposite chirality. Acid Ia and ester IIb are then converted into the desired form, i.e. ester IIa and acid Ib respectively.

For example, with reference to FIG. 2, if the acid (Ia) is the desired enantiomer, the residual ester (IIb) is first separated off (e.g. by extraction at alkaline pH) and then the desired acid is isolated (e.g. by extraction at acidic pH). If the acid (Ia) formed in the reaction (FIG. 2) is the undesired enantiomer, the residual ester (IIb, the desired enantiomer) can be directly separated (e.g. by extraction at alkaline pH). Following separation, the acid functional group (Ia) or ester functional group (IIb) of the pure enantiomers can be converted into the desired form (IIa or Ib) by simple chemical syntheses (hydrolysis, esterification).

The following examples serve to illustrate the present invention.

### EXAMPLE 1

A 500 ml three-necked flask having a KPG stirrer, pH electrode and feed for a burette is filled with 380 ml of water and 19.2 g (77.3 mmol) of (rac)-methyl 3-hydroxy-5-phenyl-3-propyl-(E)-4-pentenoate which is suspended therein with vigorous stirring and warmed to 40° C. The reaction is started by addition of 4.0 ml of Novozym® 525F. The pH is kept constant by continuous addition of 2.0 N NaOH. The end of the reaction is determined by HPLC analysis.

The pH of the enzyme-containing solution is then adjusted to 8 using 1N NaOH and the solution is then extracted 3 times using 200 ml of MTBE. The combined organic phases are dried over Na₂SO₄, filtered and concentrated under reduced pressure. The residue contains (R)-(+)-methyl 3-hydroxy-5-phenyl-3-propyl-(E)-4-pentenoate (viscous oil; yield: 9.3 g (37.4 mmol, 48%); [α]_{D}²⁰=+8.8 c=10, CHCl₃); ee=72%).

The residual alkaline aqueous solution is adjusted to pH=2 using 1N H₂SO₄ and extracted 3 times with 200 ml of MTBE. The combined organic phases are dried over Na₂SO₄, filtered and concentrated under reduced pressure. The residue contains (S)-(−)-3-hydroxy-5-phenyl-3-propyl-(E)-4-pentenoic acid (colorless powder; yield: 5.13 g (21.9 mmol, 28%); m.p.: 86-87° C., [α]_{D}²⁰=−7.7 (c=10, CHCl₃); ee=96%).

### EXAMPLES 2 TO 5

Optical resolution was carried out using the components shown in Table 1 in a manner analogous to Example 1. The selectivity indicates the efficiency of the reaction.

TABLE 1

| Substituents corr. to Formula (III) | Racemate | Enzyme | Selectivity according to Sih* |
|---|---|---|---|
| R¹ = —CH═CH—C₆H₅; R² = —CH₂—CH₃; R⁵ = —CH₃ | 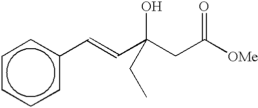 | CAL-B | 46 |
| R¹ = —CH₂—CH₂—C₆H₅; R² = —CH₂—CH₂—CH₃; R⁵ = —CH₃ | 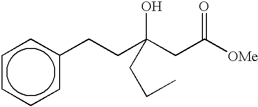 | PLE | 30 |
| R¹ = —CH₂—CH₂—C₆H₅; R² = —CH₂—CH₃; R⁵ = —CH₃ | 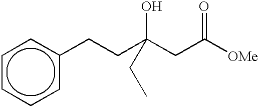 | PLE | 17 |
| R¹ = —CH₂—C₆H₅; R² = —CH₂—CH₃; R⁵ = —CH₃ | 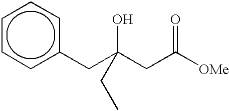 | PLE | 12 |

*C.-S. Chen et al., J. AM. CHEM. SOC. 104, 7294-7299 (1982).

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A process for the preparation of an enantiomerically pure tertiary β-hydroxycarboxylic acid having formula Ia or Ib or an enantiomerically pure tertiary β-hydroxycarboxylic acid ester having formula IIa or IIb: 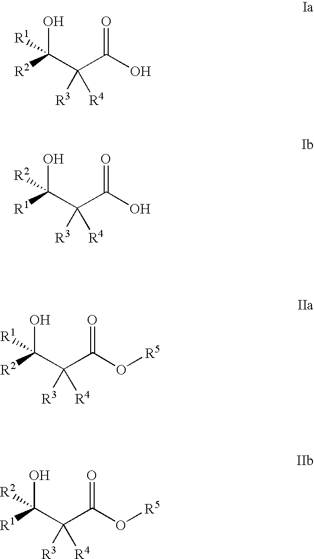
the process comprising bringing an enantiomer mixture of compounds having formulas IIa and IIb into contact with an enzyme that hydrolytically cleaves an ester bond, in an aqueous medium, such that one enantiomer of the enantiomer mixture is hydrolyzed;
wherein
R¹ and R² are each independently an optionally substituted C₆-C₁₈-aryl, C₃-C₁₈-heteroaryl, C₂-C₁₈-alkyl, C₂-C₁₈-alkenyl, C₂-C₁₈-alkynyl, C₆-C₁₈-aryl-C₁-C₁₈-alkyl, C₃-C₁₈-heteroaryl-C₁-C₁₈-alkyl, C₆-C₁₈-aryl-C₂-C₁₈-alkenyl, C₃-C₁₈-hetero-aryl-C₂-C₁₈-alkenyl, C₁-C₁₈-alkoxy-C₁-C₁₈-alkyl, C₁-C₁₈-alkoxy-C₂-C₁₈-alkenyl, C₆-C₁₈-aryloxy-C₁-C₁₈-alkyl, C₆-C₁₈-aryloxy-C₂-C₁₈-alkenyl, C₃-C₈-cycloalkyl, C₃-C₈-cyclo-alkyl-C₁-C₁₈-alkyl, or C₃-C₈-cycloalkyl-C₂-C₁₈-alkenyl group with the proviso that R¹ and R² are not the same, or R¹ and R² together with the carbon to which they are bonded form an optionally substituted or heteroatom-containing C₃-C₈-cycloalkylidene; and
R³ and R⁴ are the same or different, and are each independently an optionally substituted C₆-C₁₈-aryl, C₃-C₁₈-heteroaryl, C₁-C₁₈-alkyl, C₂-C₁₈-alkenyl, C₂-C₁₈-alkynyl, C₆-C₁₈-aryl-C₁-C₁₈-alkyl, C₃-C₁₈-heteroaryl-C₁-C₁₈-alkyl, C₆-C₁₈-aryl-C₂-C₁₈-alkenyl, C₃-C₁₈-heteroaryl-C₂-C₁₈-alkenyl, C₁-C₁₈-alkoxy-C₁-C₁₈-alkyl, C₁-C₁₈-alkoxy-C₂-C₁₈-alkenyl, C₆-C₁₈-aryloxy-C₁-C₁₈-alkyl, C₆-C₁₈-aryloxy-C₂-C₁₈-alkenyl, C₃-C₈-cycloalkyl, C₃-C₈-cycloalkyl-C₁-C₁₈-alkyl, C₃-C₈-cycloalkyl-C₂-C₁₈-alkenyl group, or R³ and R⁴ together with the carbon to which they are bonded form an optionally substituted or heteroatom-containing C₃-C₈-cycloalkylidene group, and
R⁵ is an optionally C₁-C₁₈-alkyl or C₂-C₁₈-alkenyl.

2. The process of claim 1, wherein the enzyme that hydrolytically cleaves an ester bond is a lipase or esterase.

3. The process of claim 2, wherein the enzyme that hydrolytically cleaves an ester bond is a Candida antarctica lipase type B or an esterase from porcine liver.

4. The process of claim 1, wherein the enzyme is added in an enzyme-substrate ratio calculated as the molar ratio between enzyme and substrate, of from 1:1,000 to 1:50,000,000.

5. The process of claim 1, wherein the aqueous medium is an aqueous buffer.

6. The process of claim 5, wherein an inert solvent is added to the aqueous buffer.

7. The process of claim 6, wherein the inert solvent is methyl tert-butyl ether or diisopropyl ether.

8. The process of claim 1, carried out at temperatures of 20 to 50° C.

9. The process of claim 1, wherein the reaction is stopped by separation of the unreacted enantiomer from the enantiomer mixture or from the product of the enzymatic reaction.

10. The process as claimed in claim 9, wherein the reaction is stopped by extraction of the aqueous phase or by distillation.

11. The process of claim 9, further comprising a conversion step described by Scheme 3 and Scheme 4 wherein
the acid having formula Ia is converted to the ester having formula IIa;
the acid having formula Ib is converted to the ester having formula IIb;
the ester having formula IIa is converted to the acid Ia; or
the ester having formula IIb is converted to the acid Ib:

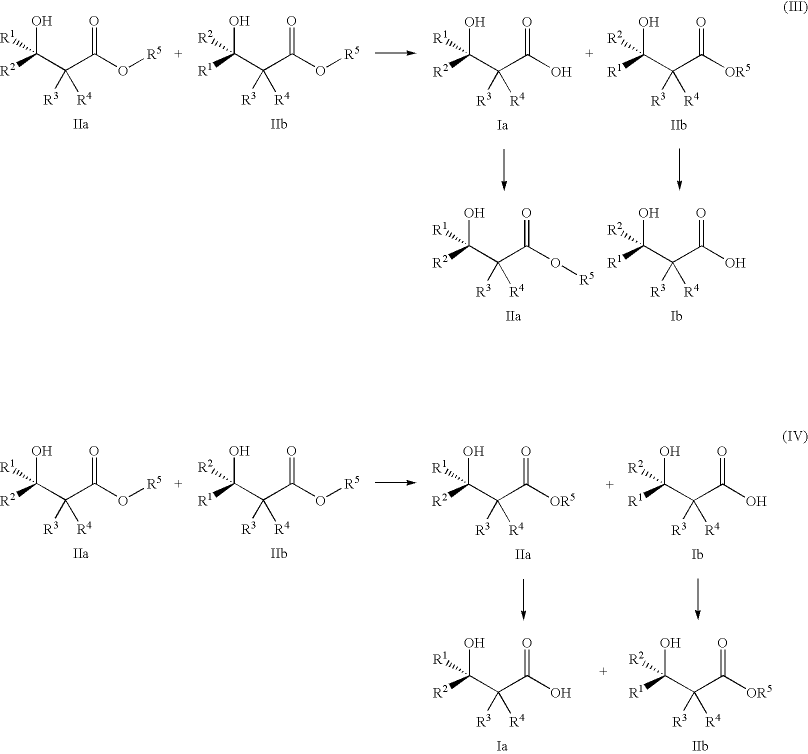

12. The process of claim 11, wherein the conversion step is hydrolysis or esterification.

13. The process of claim 1, wherein the enatiomer mixture comprises methyl 3-hydroxy-5-phenyl-3-propyl-(E)-4-pentenoate.
